(19) United States
(12) Patent Application Publication
(10) Pub. No.: US 2026/0078429 A1
(43) Pub. Date: Mar. 19, 2026

(54) SYSTEMS, METHODS, AND COMPOSITIONS FOR DETECTING EPIGENETIC MODIFICATIONS OF NUCLEIC ACIDS

(71) Applicants: Kalim MIR, Cambridge, MA (US); XGENOMES CORP., Cambridge, MA (US)

(72) Inventor: Kalim Mir, Cambridge, MA (US)

(21) Appl. No.: 18/554,150

(22) PCT Filed: Jun. 2, 2022
(86) PCT No.: PCT/US2022/032012

§ 371 Date: Oct. 5, 2023

Related U.S. Application Data
(60) Provisional application No. 63/171,566, filed on Apr. 6, 2021.

(51) Int. Cl. C12Q 1/6818

(52) U.S. Cl. CPC C12Q 1/6818

(57) ABSTRACT

Systems, methods, and compositions for detecting epigenetic modifications in nucleic acids are provided. The invention comprises methods, compositions, and systems for determining the modification status of a nucleic acid molecule by using probes to detect a difference in signal when the nucleic acid is modified compared to when it is not. The modification may comprise a covalent modification such as methylation on a nucleobase.

### CROSS-REFERENCE TO RELATED APPLICATION

The present Application claims priority to United States Provisional Patent Application no.: 63, 171,566, entitled “Detecting Epigenetic Modifications in Nucleic Acids,” filed Apr. 6, 2021, which is hereby incorporated by reference in its entirety for all purposes.

### TECHNICAL FIELD

The present disclosure is directed to systems, methods, and compounds for detecting epigenetic modifications of nucleic acids.

### BACKGROUND

Information to produce functional biological systems is written in the sequence of bases along the length of nucleic acids. The Watson-Crick double helical view of the structure of DNA does not take into account the epigenetic modifications to the nucleic acid polymer found in living organisms. A number of modifications, 5-methylcytosine (5mC), 5-hydroxymethylcytosine (5hmC), 5-formylcytosine (5fC), 5-carboxylcytosine (5caC), and N6-methadenine (6 mA), have a biological role. Of these modifications, 5-methylcytosine (5mC) is abundant in the human genome and is often called the 5th base. In contrast to the canonical bases, it is not reproduced by the cellular replication machinery and therefore not heritable in the normal way.

In mammals, methylation is most highly prevalent in the context of 5′-CpG-3′. In a double helix both Cs in the complementary CpG dyad sequence are usually methylated, although the hemi-methylated state where only one is methylated is also found and is thought to be a transitional state between a methylation and demethylation. CpG sites (CpG islands) are often found upstream of the coding regions of genes and are involved in the regulation of gene activity and tissue specific transcription.

In contrast to determining sequencing the human genome, mapping of the human methylome is a more complex task. Comprehensive, high-resolution determination of genome-wide methylation patterns from a given sample has been challenging due to the sample preparation demands and requirement to amplify or modify the nucleic acid prior to sequencing.

A variety of methods exist to isolate or detect methylated parts of the genome. The most widely used method involves treatment of DNA with bisulfite which converts unmethylated cytosine, but not 5-methyl cytosine to uracil. The DNA is then amplified (which converts all uracils into thymines) and subsequently analyzed with various methods, including microarray-based techniques and 2nd-generation (e.g. Illumina) sequencing. While bisulfite-based techniques have greatly advanced the analysis of methylated DNA, they also have several drawbacks. First, bisulfite sequencing requires a significant amount of sample preparation time. Second, the harsh reaction conditions necessary for complete conversion of unmethylated cytosine to uracil lead to degradation of DNA and thus necessitate large starting amounts of the sample, which can be problematic for some applications. Furthermore, because bisulfite sequencing relies on either microarray or 2nd generation DNA sequencing technologies for its readout of methylation status, it also suffers from the same limitations as do these methodologies.

In addition to functional modifications, modifications due to damage of DNA by a variety of agents leads to genetic mutations. In the case of RNA, while tRNAs has long been known to have a myriad of modifications, it is now increasingly appreciated that RNAs in general also have modification associated with them. Furthermore, nucleic acids can be epigenetically modified non-covalently by the binding of ligands, ranging from metals to DNA binding proteins.

Given the above-background, what is needed in the art are improved systems, methods, and compounds for detecting epigenetic modifications of nucleic acids.

### SUMMARY

The present disclosure addresses the shortcomings disclosed above by providing systems, methods, and compounds for detecting epigenetic modifications of nucleic acids.

Accordingly, one aspect of the present disclosure is directed to providing methods for directly determining the presence of epigenetic modifications on nucleic acid molecules, i.e., detection of methylation, hydroxymethylation and other modifications on DNA or RNA.

In certain aspects of the invention, methods are provided for detection of a modification in a nucleic acid molecule. In general, a sample containing a nucleic acid sequence with a possible modification and at least one probe capable of binding the nucleic acid sequence are provided. In some embodiments, the nucleic acid is bound by the probe repeatedly, and the kinetics of binding is monitored.

In some embodiments, detection of a modification in a nucleic acid molecule is done by detecting the modification directly on single molecules. In some embodiments, the detection is via a distinct signature for the modification detected on the single molecule. In some embodiments, the signature is a binding profile of one or more oligonucleotides (oligos) targeting their respective complementary sequences on the target molecule. In some embodiments, the binding profile comprises the extent (e.g. amount, speed, longevity) of hybridization to the target sequence. In some embodiments, the target molecules are immobilized on a planar surface. In some embodiments, the oligonucleotide binding is transient and repeatable. The transient binding enables super-resolution imaging (1) and the repeat binding gives confidence that a true signal is observed. Here the binding profile and extent of hybridization comprises, the number of binding events (repeat binding number) on each individual molecule, the ‘on’ or ‘dwell’ time and the “off” time (time between binding events) of these binding events. In some embodiments, the oligos are short, 7 bases or less, typically 3-5 bases in length. In some embodiments, the oligos are optically labeled (e.g., by fluorescent dyes or nanoparticles or light scattering particles) and the dwell time is the ‘bright’ time, and the off time is the ‘dark’ time.

A surprising feature discovered by the present disclosure, which forms the basis of an important embodiment of this invention, is that when the hybridization is run under conditions in which the oligo binds transiently, the bright time, dark time and/or the repeat binding number can be used to classify the binding site as being non-modified or modified and that different modifications fall into different classes. For example, in some embodiments, the dwell time of oligo binding is greater when the target sequence bears a methylation site compared to when it does not. In some embodiments, the binding profiles of multiple oligonucleotides complementary to the sequence around a base (e.g. a single base would have five 5-mers that include the base in their base footprint of binding) are taken into account to determine whether the base is modified. That multiple oligonucleotides target the same base also adds redundancy and hence robustness to the measurement.

### BRIEF DESCRIPTION OF THE DRAWINGS

FIG. 1 provides an Illustration of a method for detecting transient oligonucleotide binding to a target nucleic acid. The nucleic acid is fixed to the surface e.g. via a streptavidin/biotin interaction. An oligonucleotide probe labelled with a fluorescent dye e.g., Cy3 binds to the nucleic acid in a sequence specific manner. The oligonucleotide binds to the target in a transient manner, but long enough (e.g. 200 ms) for the fluorescent dye to be excited by a laser and the emission detected. Hybridization kinetics are measured over multiple binding and unbinding events.

FIG. 2 provides an illustrative example of the difference in binding profile kinetics between 5-MeC and non-methylated target DNA. tₒₙ is the time an oligonucleotide fluorescent signal is detected on the nucleic acid molecule. T_{off} is the time that there is no fluorescent signal detected on the nucleic acid molecule.

FIGS. 3A, 3B, 3C, 3D, 3E, 3F, 3G, 3H, 3I, and 3J collectively illustrate experimentally generated examples of oligo binding profiles for 5-MeC and non-methylated DNA targets in different contexts. Two separate flow cells are used, one containing a target nucleic acid containing 5-MeC at the indicated site on the sequence and the other flow cell containing a target nucleic acid of identical sequence but without a 5-MeC at the indicated site. The same Cy3-labelled oligonucleotide is added to each flow cell and the average tₒₙ is determined. Graphs display average tₒₙ for ten different oligonucleotides, each binding methylated and non-methylated versions of target DNA molecules.

FIGS. 4A and 4B collectively illustrate an addition of a cap such as 3′-Uaq or pyrene enables discrimination of methylated and non-methylated sites using the 3′ wobble base of the oligo. In this experimental example, it is the terminal 3′ base of the oligo that forms a base-pairing with the methylated cytosine residue in the target molecule. FIG. 4A illustrates the kinetic profiles for a single oligo binding to a methylated and non-methylated version of the same DNA target molecule. FIG. 4B illustrates the kinetic profiles for the same oligo and DNA target molecules. However, in this instance, the oligo has a 3′-Uaq cap attached.

FIGS. 5A and 5B collectively illustrate experimentally generated example of kinetic profiles for multiple oligos overlapping a single 5-MeC site, demonstrating that multiple independent readings can be taking for each position of the target molecule. Two separate flow cells are used, one containing a nucleic acid containing 5-MeC at the indicated site on the sequence and the other flow cell containing nucleic acid of identical sequence but without a 5-MeC at the indicated site. Five Cy3-labelled oligonucleotides are added to each flow cell sequentially. Graphs display average tₒₙ for each of the five overlapping oligonucleotides binding methylated and non-methylated versions of the same target DNA molecule.

FIGS. 6A and 6B collectively illustrate that, in some instances, an oligo may hybridize to a region of the target molecule that contains multiple CpG sites. These CpG sites may be either: all non-methylated, all methylated, or a combination of methylated and non-methylated. Two experimental examples are shown where these three contexts are discriminated based upon differences in oligo hybridization kinetics. FIG. 6A illustrates a non-methylated, single-methylated and double-methylated DNA target molecule containing the sequence TTCCCG is fixed to the surface and the same Cy3-labelled oligonucleotide with sequence GGGAA is added to each flow cell. FIG. 6B illustrates a non-methylated, single-methylated and double-methylated DNA target molecule containing the sequence CCCGCG is fixed to the surface and the same Cy 3-labelled oligonucleotide with sequence GCGGG is added to each flow cell. Graphs display average tₒₙ for non-methylated, single-methylated and double-methylated target molecules with each probe.

FIGS. 7A and 7B collectively illustrates a process of methylation haplotype discrimination. When an oligo hybridizes at a position on a target molecule containing more than one CpG site, its kinetic profile is determined by the methylation status of both sites (A). In the case of a mixed signal (e,g, the kinetic signal produced by hybridization to one methylated cytosine residue and one non-methylated cytosine residue), additional probes overlapping each of the CpG sites can be used to identify which are methylated and which are non-methylated (B).

FIGS. 8A, 8C, and 8C collectively illustrates detection of a spike-in and discrimination of methylation in a mixed background of cell-free DNA. An equal mixture of non-methylated and methylated synthetic single-stranded DNA targets were spiked-into synthetic plasma at a high proportion (˜50%). DNA was extracted from the mixture, biotinylated using terminal transferase and loaded onto a flow-cell. Ten oligo probes were sequentially added to identify the spike-in (8 binding and 2 not binding) and one oligo probe was added, which hybridizes the differentially methylated site. The kinetic profiles for the final probe were used determine the methylation status of each target molecule identified as spike-in (FIG. 8A). A map of all molecules detected on a portion of the flow-cell surface is shown in (FIG. 8B), with molecules identified as spike-in coloured according to their methylation status.

FIGS. 9A and 9B collectively illustrates discrimination of cytosine and hydroxymethylcytosine in a nucleic acid using 5mer oligos. The dwell time for binding of the oligo to hydroxymethylcytosine is longer than the dwell time for binding to cytosine.

FIGS. 10A and 10B collectively illustrate differentiation of cytosine (blue/dark gray), hydroxymethylcytosine (pink/red/yellow/light gray) and methylcytosine (green/purple/gray) in a nucleic acid using 4mer oligos. Experimentally generated example of oligo binding profiles for 5-hydroxymethylated (5-hmC), 5-methylated and non-methylated DNA. FIG. 10A illustrates three separate flow cells used, one containing a target nucleic acid containing two 5-hmC residues at the indicated sites, one containing two 5-MeC at the indicated sites, and the other containing a DNA target of identical sequence but without either epigenetic modification. The same Cy3-labelled oligonucleotide was added to each flow cell and the average tₒₙ is determined. FIG. 10B displays the average tₒₙ for individual molecules, colored by the type or absence of epigenetic modification. The dwell time for binding of the oligo to cytosine is shorter than hydroxymethylcytosine which is shorter than methylcytosone.

FIG. 11 illustrate an exemplary system topology including a computer system, in accordance with an exemplary embodiment of the present disclosure.

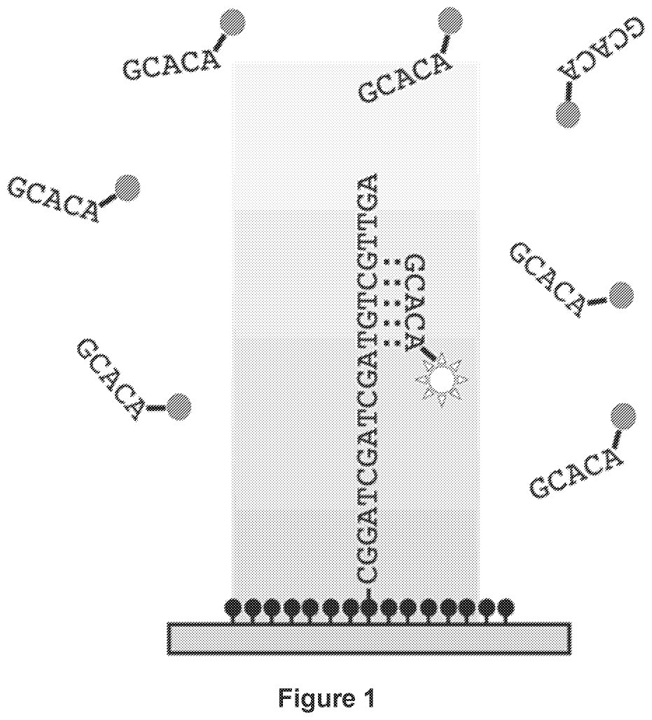

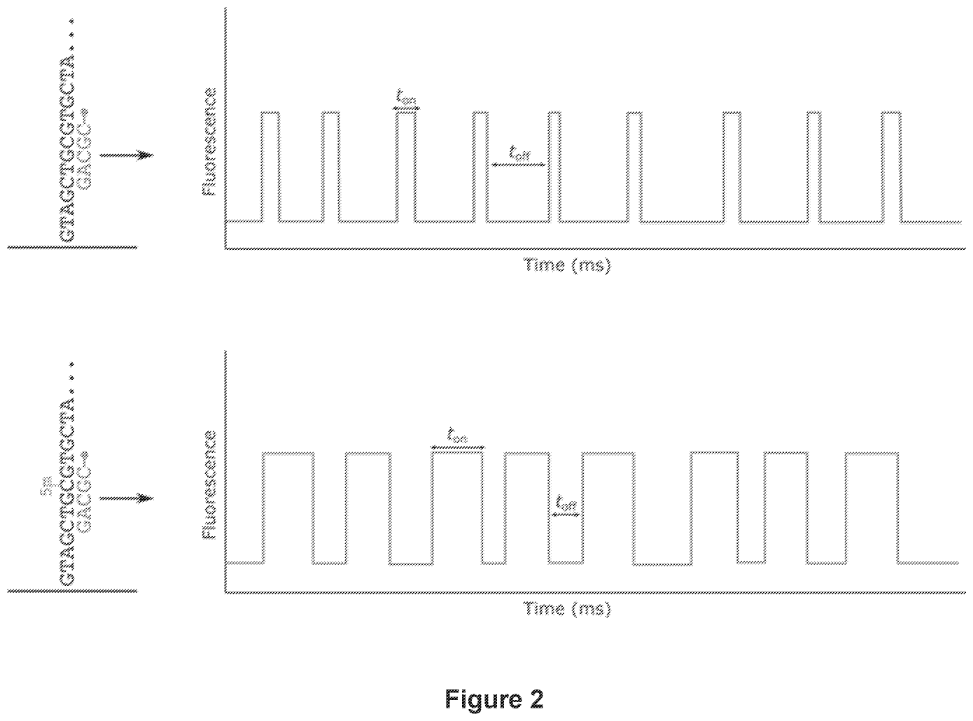

It should be understood that the appended drawings are not necessarily to scale, presenting a somewhat simplified representation of various features illustrative of the basic principles of the invention.

### DETAILED DESCRIPTION

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of +20%, +10%, +5%, or +1% of a given value. Where particular values are described in the application and claims, unless otherwise stated, the term “about” means within an acceptable error range for the particular value. The term “about” can have the meaning as commonly understood by one of ordinary skill in the art. The term “about” can refer to +10%. The term “about” can refer to +5%.

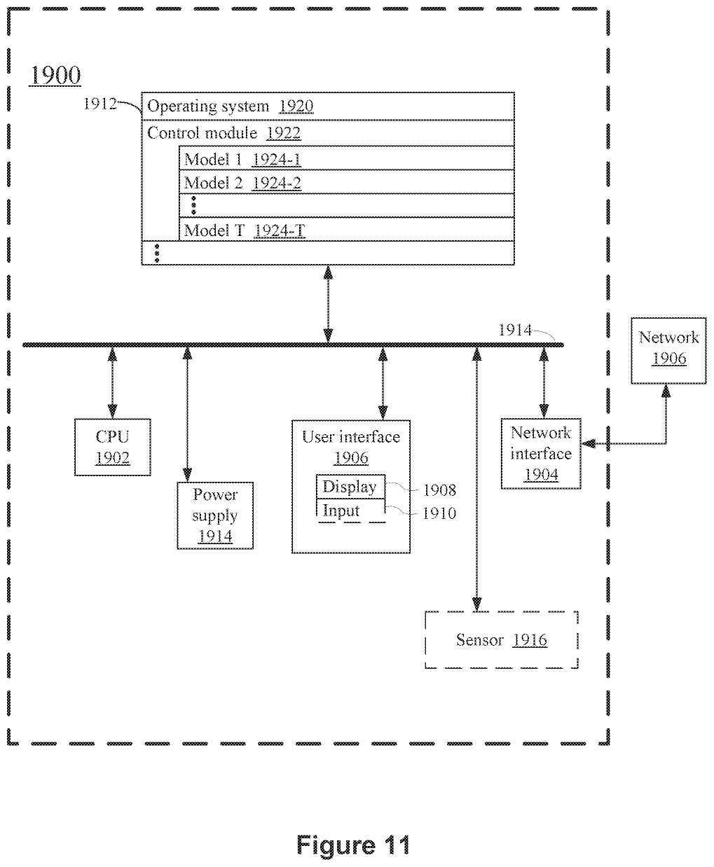

In the present disclosure, unless expressly stated otherwise, descriptions of devices and systems will include implementations of one or more computers. For instance, and for purposes of illustration in FIG. 11, a computer system 1900 is represented as single device that includes all the functionality of the computer system 1900. However, the present disclosure is not limited thereto. For instance, in some embodiments, the functionality of the computer system 1900 is spread across any number of networked computers and/or reside on each of several networked computers and/or by hosted on one or more virtual machines and/or containers at a remote location accessible across a communications network (e.g., communications network 1906 of FIG. 11). One of skill in the art will appreciate that a wide array of different computer topologies is possible for the computer system 1900, and other devices and systems of the preset disclosure, and that all such topologies are within the scope of the present disclosure. Moreover, rather than relying on a physical communications network 1906, the illustrated devices and systems may wirelessly transmit information between each other. As such, the exemplary topology shown in FIG. 11 merely serves to describe the features of an embodiment of the present disclosure in a manner that will be readily understood to one of skill in the art.

FIG. 11 depicts a block diagram of a distributed computer system (e.g., computer system 1900) according to some embodiments of the present disclosure. The computer system 1900 at least facilitates communicating one or more instructions for detecting epigenetic modifications of nucleic acids.

In some embodiments, the communication network 1906 optionally includes the Internet, one or more local area networks (LANs), one or more wide area networks (WANs), other types of networks, or a combination of such networks.

Examples of communication networks 1906 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11 g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 1900 includes one or more processing units (CPUs) 1902, a network or other communications interface 1904, and memory 1912.

In some embodiments, the computer system 1900 includes a user interface 1906. The user interface 1906 typically includes a display 1908 for presenting media. In some embodiments, the display 1908 is integrated within the computer systems (e.g., housed in the same chassis as the CPU 1902 and memory 1912). In some embodiments, the computer system 1900 includes one or more input device(s) 1910, which allow a subject to interact with the computer system 1900. In some embodiments, input devices 1910 include a keyboard, a mouse, and/or other input mechanisms. Alternatively, or in addition, in some embodiments, the display 1908 includes a touch-sensitive surface (e.g., where display 1908 is a touch-sensitive display or computer system 1900 includes a touch pad).

In some embodiments, the computer system 1900 presents media to a user through the display 1908. Examples of media presented by the display 1908 include one or more images (e.g., user interface on display 1908 presenting a chart of 3C, etc.), a video, audio (e.g., waveforms of an audio sample), or a combination thereof. In typical embodiments, the one or more images, the video, the audio, or the combination thereof is presented by the display 1908 through a client application. In some embodiments, the audio is presented through an external device (e.g., speakers, headphones, input/output (I/O) subsystem, etc.) that receives audio information from the computer system 1900 and presents audio data based on this audio information. In some embodiments, the user interface 1906 also includes an audio output device, such as speakers or an audio output for connecting with speakers, earphones, or headphones.

Memory 1912 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 1912 may optionally include one or more storage devices remotely located from the CPU(s) 1902. Memory 1912, or alternatively the non-volatile memory device(s) within memory 1912, includes a non-transitory computer readable storage medium. Access to memory 1912 by other components of the computer system 1900, such as the CPU(s) 1902, is, optionally, controlled by a controller. In some embodiments, memory 1912 can include mass storage that is remotely located with respect to the CPU(s) 1902. In other words, some data stored in memory 1912 may in fact be hosted on devices that are external to the computer system 1900, but that can be electronically accessed by the computer system 1900 over an Internet, intranet, or other form of network 106 or electronic cable using communication interface 1904.

In some embodiments, the memory 1912 of the computer system 1900 stores:
an operating system 1920 (e.g., ANDROID, IOS, DARWIN, RTXC, LINUX, UNIX, OS X, WINDOWS, or an embedded operating system such as VxWorks) that includes procedures for handling various basic system services;
an electronic address associated with the computer system 1900 that identifies the computer system 1900 (e.g., within the communication network 1906);
a control module 1922 including one or more modules 1924 for controlling one or more processes (e.g., method) associated with the computer system 1900; and
optionally, a client application for presenting information (e.g., media) using a display 1908 of the computer system 1900.

In some embodiments, the control module 1922 includes one or more models 1924 that is configured to perform one or more steps of a method of the present disclosure (e.g., a first method for determining a methylation status of at least a portion of a nucleic acid molecule, a second method for determining a modification status of multiple nucleic acid molecules encompassing different sequences, a third method for determining a state of modification (such as methylation) of one or more nucleic acid molecules, a fourth method for determining a sequence and episequence of at least a portion of a nucleic acid molecule, etc.).

Furthermore, in some embodiments, the computer system includes one or more reference libraries (e.g., one or more reference databases, such as a cancer reference database, a nucleic acid reference database, etc.).

Modifications detectable by the methods provided herein include chemically modified bases, enzymatically modified bases, DNA lesions, abasic sites, non-natural bases, secondary structures, and agents bound to a nucleic acid. Exemplary modifications that can be detected by the methods of the invention include, but are not limited to methylated bases (e.g., 5-methylcytosine, N6-methyladenosine, etc.), pseudouridine bases, 7,8-dihydro-8-oxoguanine bases, 2′-O-methyl derivative bases, nicks, apurinic sites, apyrimidic sites, pyrimidine dimers, a cis-platen crosslinking products, oxidation damage, hydrolysis damage, bulky base adducts, thymine dimers, photochemistry reaction products, interstrand crosslinking products, mismatched bases, secondary structures, and bound agents.

In some embodiments, the binding characteristics of the oligos are tuned by using different modifications in the probes, for example there are several options for increasing the binding stabilities of short oligos (LNA, PNA, Locked Nucleic Acid (LNA), Peptide Nucleic Acid (PNA), analog bases with altered stability and minor-groove binding, stacking, intercalating, cationic conjugates, etc.) and certain modifications are able to accentuate the difference in binding between modified and non-modified bases. In some embodiments, the binding characteristics are tuned by buffer composition, particularly the concentration and type of salt, the presence of denaturants, binding accelerators, pH, temperature and oligo probe concentration.

In some embodiments, in order to measure the binding of oligos the target must be fixed to a surface so that measurements (and their repetition) for determining identity and methylation status can be carried out on the same molecule.

In some embodiments, where the sequence or identity of the target molecule is already known or specified, the invention involves determining only the modification status of the target molecules.

In some embodiments, the method includes binding one or more oligos to the nucleic acid molecule that bind differently to a sequence if it is modified compared to when it is not.

In some embodiments, where the identity of the target molecule is not previously known, for example when random or shotgun fragments of genomic DNA are interrogated detecting an epigenetic modification in such a nucleic acid sample requires the determination of the identity of the nucleic acid molecule as well, thus this embodiment comprising two main aspects. In some embodiments, a first main aspect includes obtaining sequence information from the nucleic acid molecule to determine its identity. In some embodiments, a second main aspect includes binding one or more oligos to the nucleic acid molecule that bind differently to a sequence if it is modified compared to when it is not.

In some embodiments, the sequence information is obtained by sequencing, e.g. PacBio sequencing, Helicos sequencing, Oxford nanopore sequencing, XGenomes sequencing (2,3). In some embodiments, the sequence information is obtained by molecular probing (2,3)

In some embodiments, the method of detecting an epigenetic modification in a nucleic acid sample comprises two main aspects. In some such embodiments, a first aspect includes binding one or more oligos to the nucleic acid molecule to determine its identity. In some such embodiments, a second aspect includes binding one or more oligos to the nucleic acid molecule that bind differently to a sequence if it is modified compared to when it is not.

In some embodiments, an assigned identity is based upon determining if one, or which of more than one oligos bind to the target and what their binding profile (repeat binding number, bright time, dark time) looks like.

In some embodiments, identity of a nucleic acid comprises its genomic origin.

In some embodiments, the modification status is determined by matching the binding profile of one or more oligos to the expected binding profiles when one or more modifications would be present. In some embodiments, if the identity has been determined, it enables data for molecules whose modification status is of interest to be selectively processed (e.g., processed by a model 1924 of control module 1922 of FIG. 11).

In some embodiments, historical or training data (e.g., historical or training data of a model 1924 of control module 1922 of FIG. 11) is used to determine whether an acquired binding profile corresponds to the presence of a modification or not.

In some embodiments, spiked-in controls are used as a reference to determine whether an acquired binding profile corresponds to the presence of a modification or not.

In some embodiments, the extent of binding of the probe to a modified complementary sequence and non-modified complementary sequence has been previously determined or is determined in situ by observing the binding to reference spiked-in targets.

In some embodiments, spiked in controls allow setting of a normal signal level for the modified bases and the non-modified bases. For example, such controls comprise one or more synthetic oligonucleotides bearing modified bases in particular sequence contexts and their sequence-matched oligonucleotides with no modified base.

In some embodiments, the binding comprises duplex formation. Hence, in some embodiments, the present disclosure provides a method for determining the methylation status of at least a portion of a nucleic acid molecule.

In some embodiments, the method includes measuring the extent of hybridization of a complementary oligonucleotide probe to a test target sequence.

In some embodiments, the method includes optionally measuring the extent of hybridization of a different complementary oligonucleotide probe to a test target sequence

In some embodiments, the method includes deciding that methylation is present if the extent of hybridization is greater than the extent of hybridization to a reference non-methylated target sequence and/or is equivalent to a reference methylated target sequence.

In some embodiments, the pattern of hybridization of two or more oligonucleotides to the target sequence is obtained.

In some embodiments, the present disclosure is directed to providing a method for determining the modification status of multiple nucleic acid molecules encompassing different sequences.

In some such embodiments, the method includes obtaining a sample of nucleic acid molecules.

In some such embodiments, the method includes dispersing and immobilizing/fixing the nucleic acid molecules on a surface, thus obtaining an array of nucleic acid molecules within which array each molecule is fixed at a distinct location on the surface.

In some such embodiments, the method includes exposing one or more oligos (typically a repertoire or panel of oligos) of known sequence to the nucleic acids, one or more of said oligos capable of determining the identity of each individual nucleic acid molecule and detecting the binding of one or more of said oligos to each individual nucleic acid and determining the identity of the said nucleic acid.

In some such embodiments, the method includes exposing one or more oligos of known sequence to the nucleic acids, one or more of said oligos capable of having a different binding profile when the sequence is modified compared to when the sequence is not modified and detecting the binding profile of one or more said oligos to each individual nucleic acid and determining if the binding profile better matches the binding profile of when the sequence is modified or the binding profile of when the sequence is not modified.

In some such embodiments, the method includes recording the modification status of the identified molecules.

In some embodiments, the present disclosure is directed to providing a method for determining the modification status of multiple nucleic acid molecules encompassing different sequences.

In some embodiments, the method includes obtaining a sample of nucleic acid molecules.

In some embodiments, the method includes dispersing and immobilizing/fixing the nucleic acid molecules on a surface, thus obtaining an array of nucleic acid molecules within which array each molecule is fixed to the surface.

In some embodiments, the method includes exposing one or more oligos (typically a repertoire or panel of oligos) of known sequence to the nucleic acids, one or more of said oligos capable of determining the identity of each individual nucleic acid molecule and one or more of said oligos having a different binding profile when the sequence of the nucleic acid molecule is modified compared to when the sequence is not modified.

In some embodiments, the method includes detecting the binding of one or more of said oligos to each individual nucleic acid and determining the identity of the said nucleic acid and detecting the binding profile of one or more said oligos to each individual nucleic acid and determining if the binding profile better matches the binding profile of when the sequence is modified or the binding profile of when the sequence is not modified.

In some embodiments, the method includes recording the modification status of the identified molecules.

In some embodiments, the present disclosure is directed to providing a method of determining the state of modification (e.g., methylation) of one or more nucleic acid molecules.

In some embodiments, the method includes exposing one or more oligos of known sequence to the nucleic acids

In some embodiments, the method includes detection of whether one or more oligos has hybridized to any of the nucleic acid molecules

In some embodiments, the method includes exposing the nucleic acid molecules to one or more oligos of known sequence and known hybridization behavior with respect to a modification of nucleic acids, such as methylation

In some embodiments, the method includes detection of whether one or more oligos has hybridized to any of the nucleic acid molecules

In some embodiments, the method includes building a binding profile for each nucleic acid molecule. The binding profile comprises a set of one or more binding calls for each oligo that was exposed to the nucleic acid molecules. Moreover, the binding profile comprises, a confidence metric for each binding call that captures an estimation of the likelihood of error for that call.

In some embodiments, the method includes set of one or more reference nucleic acid sequences.

In some embodiments, the method utilizes a computer database (e.g., computer system 1900 of FIG. 11) that stores positions of exact matches between one or more oligonucleotide sequences and one or more of the reference sequences.

In some embodiments, the method utilizes a computer program (e.g., control module 1922 of computer system 1900 of FIG. 11) that uses the database of exact matches among the one or more reference sequences to convert each binding profile into one or more intervals within any subset of reference sequences that are most likely to encompass the nucleic acid sequence that corresponds to the nucleic acid molecule.

In some embodiments, the method utilizes a computer program (e.g., control module 1922 of computer system 1900 of FIG. 11) that uses the one or more matching intervals among the one or more reference sequences and the subset of the binding profile that corresponds to oligonucleotides that are sensitive to modifications to build a modification profile for each of the nucleic acid molecules.

In some embodiments, the molecules are dispersed on a surface such that on average they are located<250 nanometers (nm) apart on the surface and are resolved by super-resolution imaging.

In some embodiments, the binding events on the individual molecules are localized by single molecule localization.

The same approaches as the above can be used for a range of modifications, although the extent of binding may be greater or lesser than unmodified nucleic acid depending on the modification.

Methylation is far the most prevalent modification in the genome but in a real-world sample, other modifications will co-exist in the genome, therefore a means to distinguish and classify different modifications is useful. Because of this complexity, in some embodiments, if only information about a particular modification is needed, then that modification may be tagged and the binding profile to the tag is measured. For example, β-glucosytransferase (BGT) can tag hydroxymethyl to provide a pronounced signal which can be detected.

In some embodiments, there may be multiple modifications within the footprint of a single oligo along the target sequence, this will affect the binding profile. Historical and training data (e.g., historical and training data of a model 1924 of control module 1922 of FIG. 11) can be used to differentiate whether there is one, two or three CpG modifications in the footprint of a 5-mer. The aggregate data from multiple oligos targeting the locality will also aid this determination. In some other cases there may be modifications of different types within the footprint of an oligonucleotide. For example, methyl and hydroxymethyl sites may be found in the same locality. Similarly, historical and training data can be used to determine what type of modifications are present. In both cases, well-chosen spiked-in controls are used to aid the determination.

In some embodiments, machine learning can be used to determine a binding profile with different numbers of modifications or type of modification.

In some embodiments, the measurements provide an estimate, nonetheless the estimate is pertinent to judging the status of modification and how it is likely to affect a biological process or medical condition. In many cases the modification status is used as a biomarker and it may be one biomarker among several, that in combination are used to provide a likelihood and hence a clinical decision or be the basis of hypothesis about a molecular phenomenon.

In some embodiments, a termini of sample DNA molecules are modified to facilitate immobilization and fixing on the surface. In some embodiments, a termini is modified by using terminal transferase to add one or more nucleotides. In some embodiments, a single modified nucleotide is added using terminal transferase. In some embodiments, a homopolymers is added using deoxynucleotides. Some of the nucleotides are modified to facilitate capture onto a surface, e.g. modified with biotin for immobilization onto a streptavidin/neutravidin surface or modified with amino-allyl for immobilization onto a-COOH surface. In some embodiments, ligation is used to add a short oligo to the termini, the oligo may bear a modification to facilitate capture onto a surface. In some embodiments, the oligonucleotide or homopolymer is hybridized to a complementary sequence attached to a surface and the target sequence is thus immobilized.

In some embodiments, the more than one oligos is bound in multiple cycles. In some embodiments, there is one or more a wash step(s) between the binding of one oligo and another. In some embodiments, multiple oligos are bound and exposed (multiplexed) in one cycle. In some embodiments, the oligos are labeled with the same label (e.g. a fluorescent or light scattering or plasmon resonating label). In some embodiments, the oligos are labeled with a different label. In some embodiments, the different labels are represented by different wavelengths of emission and/or excitation. In some embodiments, the different labels are represented by different physical properties comprising fluorescence lifetime, anisotropy, optical permitivity.

In some embodiments, a label is at one end of the oligonucleotide. In some embodiments, there is a label at both ends of the oligonucleotide. In some embodiments, there is a label internally in the oligonucleotides

In some embodiment, a methylation sensitive reagent (e.g. antibody or modification binding protein or other ligand) occupies a site where a modified nucleotide is present and modulates the binding of oligonucleotides. The oligonucleotide may be complementary to the site occupied by the methylation sensitive reagent.

In some embodiments, the identification and modification status of each of the molecules is aggregated to provide an insight into biological process or medical condition.

In some embodiments, the degree of modification per target molecule is estimated. In some embodiments, a modification haplotype is determined.

In some embodiments, duplex DNA is denatured before or after immobilization so that the molecules that are interrogated are single strands.

The method does not need the modification to be modified (e.g. β-glucosytransferase (BGT) labeling of hmC is not required) nor does it need separate sequencing of the treated and untreated portion of the sample e.g. bisulfite sequencing for methylation detection, and it does not need an amplification step such as the polymerase chain reaction.

Nevertheless, in some embodiments, the differential oligonucleotide binding methods of this invention can discriminate between base modified intermediates of common methylation/hydroxymethylation kits including Tet-assisted Pyridine-Borane sequencing (TAPS, Base Genomics/Exact Sciences) and Enzymatic methyl-seq (New England Biolabs).

In some embodiments, individual nucleic acid molecules are attached to a surface as part of an array of nucleic acid molecules. In some embodiments, the array comprises molecules of a range of different species or sequences (e.g., fragments comprising a complete transcriptome or a whole human genomes). Many molecules in the array may fully or partly share sequence. In some embodiments, the array is a single molecule array. Yhe sample molecules are arrayed at random but at distinct locations on the surface. In some embodiments, the molecules remain fixed at the distinct locations throughout the molecule identification and modification detection process. In some embodiments, the distinct location at which a particular molecule is attached is not known before the identity of the molecule is determined as part of the method of the invention.

In some embodiments, the identity of the target molecule is already known and only the modification status of the molecules is determined. The modification status may include the pattern of modifications along the target sequence. In some embodiments, the target molecules form part of a spatially addressable array or microarray. In some such embodiments, the identity of the molecule in each element/spot of the microarray is known. In some embodiments, the microarray elements/spots contain multiple molecules and the modification status is determined as a bulk measurement. In some embodiments, the microarray spots contain multiple molecules and the modification status is determined for individual molecules in the element/spot.

In some embodiments, the target molecules are not attached to a substrate but are free in solution. In some such embodiments, the target molecule is single stranded. In some such embodiments, the epigenetic status is determined by adding the epigenetic modification detection oligonucleotide probes into the solution and then measuring melting curves. The modification is detected by the need for a different (higher for the case of hydroxymethyl C and methyl C) temperature to melt the heteroduplex formed between the target molecule and the probes when there is a modification present compared to when there is no modification present.

In some embodiments, the present disclosure is directed to providing a method of determining a sequence and episequence of at least a portion of a nucleic acid molecule.

In some embodiments, the method includes fixing the nucleic acid molecule on a test substrate when the nucleic acid molecule is a single stranded molecule or denaturing the nucleic acid molecule to a single stranded molecule and fixing the single stranded nucleic acid molecule on the test substrate when the nucleic acid molecule is a double stranded molecule or fixing the nucleic acid molecule on the test substrate and denaturing the nucleic acid molecule on the test substrate to a single stranded molecule when the nucleic acid molecule is a double stranded molecule, thereby forming a fixed single stranded nucleic acid on the test substrate.

In some embodiments, the method includes exposing the fixed single stranded nucleic acid to a respective oligonucleotide probe species in a set of oligonucleotide probe species. Each respective oligonucleotide probe species of the set of oligonucleotide probe species is capable of hybridizing to its complementary portion located at one or more locations on the fixed single stranded nucleic acid and has: (i) a unique respective predetermined sequence, (ii) a predetermined length, and (iii) a respective label selected from the group consisting of a dye, a fluorescent nanoparticle, a plasmon resonant particle, a light-scattering particle, a nanoparticle, and a fluorescence resonance energy transfer (FRET) partner (which is capable of producing a fluorescent signal). The exposing step occurs under conditions such that: i) oligonucleotide probes of the respective oligonucleotide probe species of the set of oligonucleotide probe species repetitively transiently and reversibly bind to the one or more locations on the fixed single stranded nucleic acid on the test substrate, thereby forming a respective transient heteroduplex on each of the one or more locations on the fixed single stranded nucleic acid on the test substrate; and ii) respective instances of optical activity from the respective label are generated detected by repetitively transiently and reversibly binding the oligonucleotide probes of the respective oligonucleotide probe species of the set of oligonucleotide probe species to the one or more locations on the fixed single stranded nucleic acid on the test substrate and are detected at each of the one or more locations on the fixed single stranded nucleic acid on the test substrate.

In some embodiments, the method includes determining if one or more portions of the fixed single stranded nucleic acid are complementary to the respective oligonucleotide probe species of the set of oligonucleotide probe species by counting and measuring the duration of respective instances of optical activity on each of the one or more locations on the fixed single stranded nucleic acid on the test substrate occurring during the exposing step using a two-dimensional imager capable of detecting the respective instances of optical activity generated from the respective label, thereby obtaining a first set of one or more positions on the fixed single stranded nucleic acid that are complementary to the respective oligonucleotide probe species of the set of oligonucleotide probe species.

In some embodiments, the method includes washing the test substrate to remove the respective oligonucleotide probe species of the set of oligonucleotide probe species from the test substrate.

In some embodiments, the method includes repeating the exposing step, measuring step, and washing step by exposing the fixed single stranded nucleic acid on the test substrate to the another respective oligonucleotide probe species in the set of oligonucleotide probe species, thereby obtaining a second set of one or more positions on the fixed single stranded nucleic acid that are complementary to another respective oligonucleotide probe species in the set of oligonucleotide probe species.

In some embodiments, the method includes determining the sequence of at least the portion of the nucleic acid based at least in part on the first set of one or more positions on the fixed single stranded nucleic acid that are complementary to the respective oligonucleotide probe species of the set of oligonucleotide probe species and the second set of one or more positions on the fixed single stranded nucleic acid that are complementary to the another respective oligonucleotide probe species of the set of oligonucleotide probe species.

And determining whether the portion of the nucleic acid molecule has one or more epigenetic modifications based on an observed differential binding behavior of the oligonucleotide probes of the respective oligonucleotide probe species in the set of oligonucleotide probe species to their complementary portion located at one or more locations on the fixed single stranded nucleic acid when the one or more locations has an epigenetic modification compared to when the one or more locations does not have an epigenetic modification.

In some embodiments, the differential binding behavior comprises a difference in repeat binding number obtained through counting the instances of optical activity), on rate, dwell time. In some embodiments, the differential binding behavior comprises a difference in repeat binding number, dark time (when there is no detectable optical activity) and/or bright time (when there is optical activity).

In some embodiments, the binding kinetics of probes (e.g. bright time, dark time, number of repeat binding events) is used to determine the methylation status of cytosines in each fragment by either or both of: (i) comparing the probe binding data to a database (e.g., computer system 1900 of FIG. 11) of previously collected data from probes binding unmodified and modified cytosines; and/or (ii) comparing the probe binding data for each DNA fragment to the probe binding dynamics for that probe for all DNA fragments in the sample (and/or dynamics of probes binding control DNA spiked into the sample).

In some embodiments, the binding profiles of each oligonucleotide capable of binding to a nucleic acid sequence that may bear a modification are characterized in advance by testing against synthetic modified and non-modified versions of the nucleic acid sequences that may bear a modification, and thus serve as a reference to compare the binding profiles obtained for the sample molecules.

In some embodiments, the information regarding the modification status of sample molecules obtained by the methods of the invention is used as the basis for determining a biological state or a medical condition.

### Compositions

In some embodiments, compositions for oligonucleotides of known sequence to be used in the invention. Some embodiments comprise oligonucleotides of <8, <7, <6, <5, <4 nucleotides in length. In some embodiments, the composition comprises a repertoire or panel of oligonucleotides all of the same length. In some embodiments, the length of the oligos are different. In some embodiments, the oligos comprise LNA nucleotides. In some embodiments, the oligos comprise LNA/DNA oligos. In some embodiments, the oligos comprise DNA, LNA, LNA/DNA oligos, of one or more lengths. In some embodiments, one or more positions on the oligonucleotide is methylated. In some embodiments, the modification is at the 5 position on the base. In some embodiments, some of the oligos comprise a non-defined N or universal base position. In some embodiments, some of the oligos comprise a conjugate. In some embodiments the conjugate is a ZNA or spermine residues or other positively charged residues, In some embodiments, the conjugates are, intercalating or stacking/capping structures. In some embodiments, the capping structures comprise UAQ (e.g. attached via reagent: 5′-Dimethoxytrityl-Uridine, 2′-(anthraquinone-2-yl-carboxamido)-3′-succinoyl-long chain alkylamino-CPG), Pyrene, Thiazole Orange. In some embodiments, some of the probes comprise multiple copies of the oligos linked together. In some embodiments, copies of the probe sequences are connected tandemly with or without a spacer (e.g. hexatheylene glycol), in one such embodiment a label is attached to one of the nucleosides. In some embodiments, the probes are connected via branching amidite to for a dendrimer, each probe is an arm or branch of the dendritic structure. In some embodiments, the label is at one branch of the dendrimer. In some embodiments, the label is at multiple branches of the dendrimer. In some embodiments, some of the probes are PNA or other non-native backbone. In some embodiments, some of the bases are modified to increase duplex stability. In some embodiments, some of the bases are modified to increase nucleation ability. In some embodiments, some of the bases are modified to decrease duplex stability. In some embodiments, the conjugates are at one end. In some embodiments, the conjugates are at both ends. In some embodiments, the conjugates are internal. Some embodiments comprise compositions for buffers that are effective for the invention: TMACl, SSC, Ethylene Carbonate, Dextran sulfate, Formamide, PEG, Urea, Betaine, etc.

RNA modifications, including N6-methyladenosine (m6A), N6,2′-O-dimethyladenosine (m6Am), 8-oxo-7,8-dihydroguanosine (8-oxoG), pseudouridine (Y), 5-methylcytidine (m5C), and N4-acetylcytidine (ac4C) are amenable to the methods of this invention.

DNA modifications, including 5-methylcytosine (5mC), 5-hydroxymethylcytosine (5hmC), 5-formylcytosine (5fC), and 5-carboxylcytosine (5caC) are amenable to the methods of this invention.

Furthermore, additional details and information regarding the systems, methods, and compounds of the present disclosure can be found at United States Patent Publication no.: 2019/0149681 A1, entitled “Image Processing System, Image Processing Apparatus, Control Method of Imaging Processing Apparatus, and Program,” published May 16, 2019; Jungmann et al., 2010, “Single-molecule kinetics and super-resolution microscopy by fluorescence imaging of transient binding on DNA origami.” Nano letters, 10 (11), pg. 4756-4761; and United States Patent Publication no.: 2022/0064712 A1, entitled “Sequencing by emergence,” published Mar. 3, 2022; United States Patent Publication no.: 2020/0082913 A1, entitled “Systems and Methods for Determining Sequencing,” published Mar. 12, 2020; United States Patent Publication no.: 2020/0056229 A1, entitled “Sequencing by emergence,” published Feb. 20, 2020; U.S. Pat. No. 10,982,260 B2, entitled “Sequencing by emergence,” issued Apr. 20, 2021; each of which is hereby incorporated by reference in its entirety.

### EXAMPLES

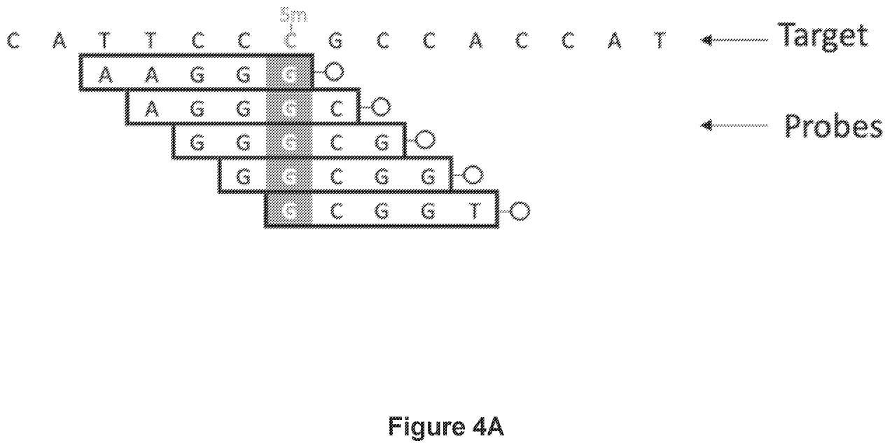

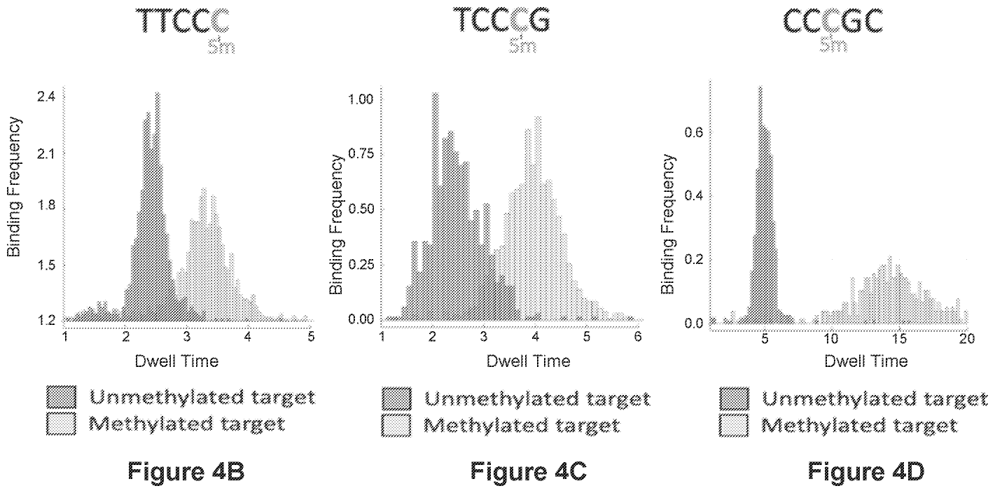

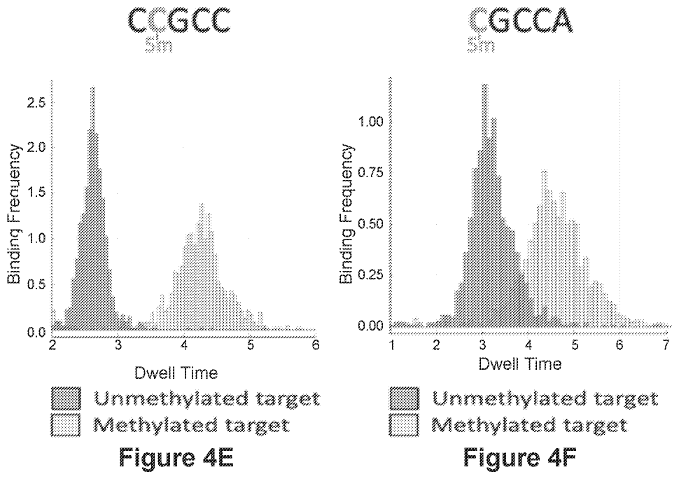

### Example 1: Detection of Methylated Single Nucleic Acid Molecule (FIG. 4)

A nucleic acid single stranded DNA molecule was synthesized with the sequence

Target 1:
Biotin-TTTTTTTTTTTTTTTTTTTTTTTTTTTTTCCATTCCCGCCAC
CATCGCCTCAATCCCTGTGCGCTAATTTTTTTTTTTTTTTTTTTTTTTT
and methylated version was synthesized
Target 2:
Biotin-TTTTTTTTTTTTTTTTTTTTTTTTTTTTTCCATTCccGCCAC
CATcGCCTCAATCcCTGTGCGCTAATTTTTTTTTTTTTTTTTTTTTTTT.

A custom flow cell was prepared using a streptavidin coated cover glass attached to plastic flow cell chamber. The flow cell was washed with BX (5 mM Tris, 10 mM MgCL2, 0.05% Tween 20 and 1 mM EDTA). 10 pM of Target 1 and Target 2 was added to separate chambers of the flow cell. After 5 min the flow cell was washed with 150 ul of BX. 5 nM of oligonucleotides (Cy3-GGTAG, Cy3-GTAGC, Cy3-TAGCG, Cy3-AGGTA, Cy3-GGTGG, Cy3-CGGAG) in BX+F (5 mM Tris, 10 mM MgCL2, 0.05% Tween 20 and 1 mM EDTA, 30% Formamide) was added to each flow cell sequentially. Washing between oligonucleotide imaging was performed with 150 ul BX+F three times. Imaging was performed on the ONI Nanoimager S in TIRF mode with the focus lock activated and 10% laser (532 nm) power. A total of 2000 (200 alternatively as few as 200 or as numerous as up to 8000) frames with 200 ms (alternatively as low as 25 ms) per frame was captured per oligo.

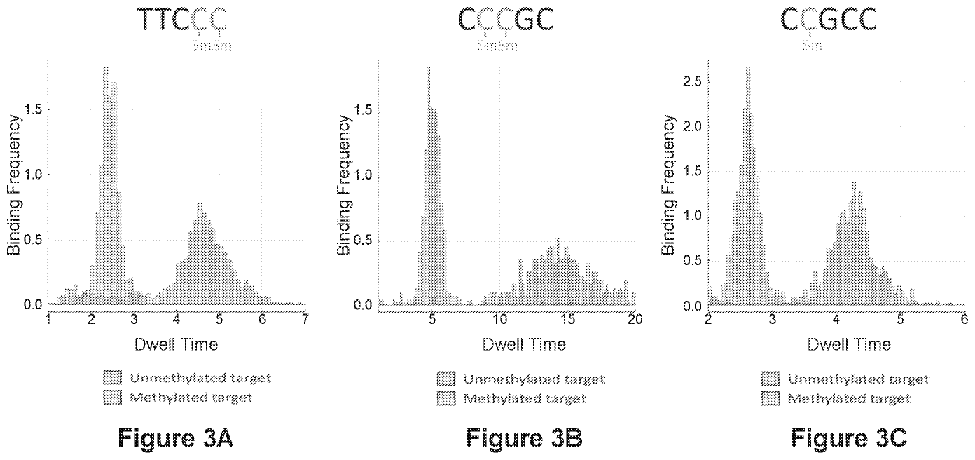

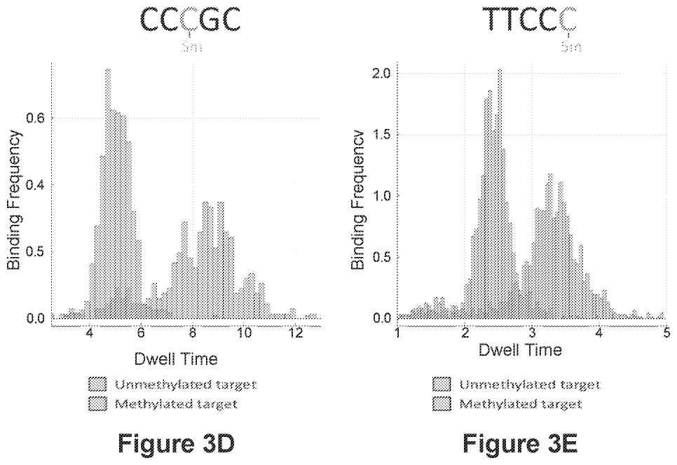

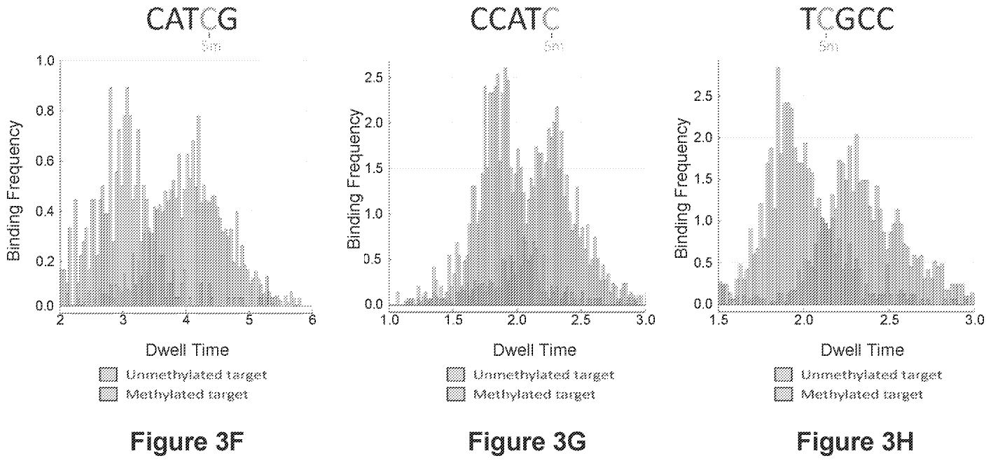

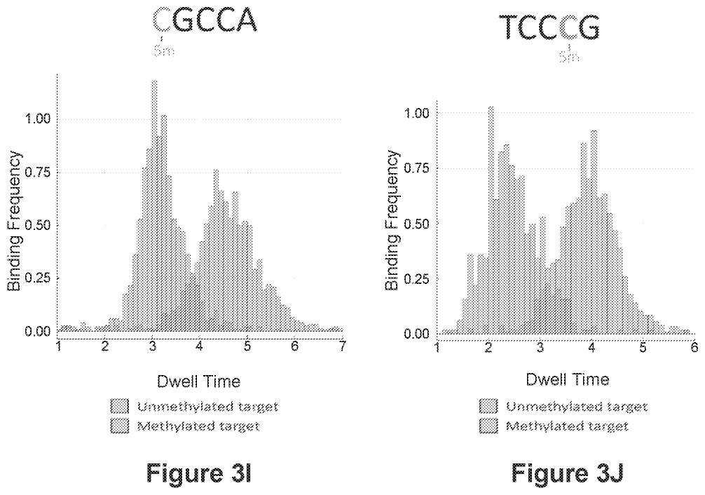

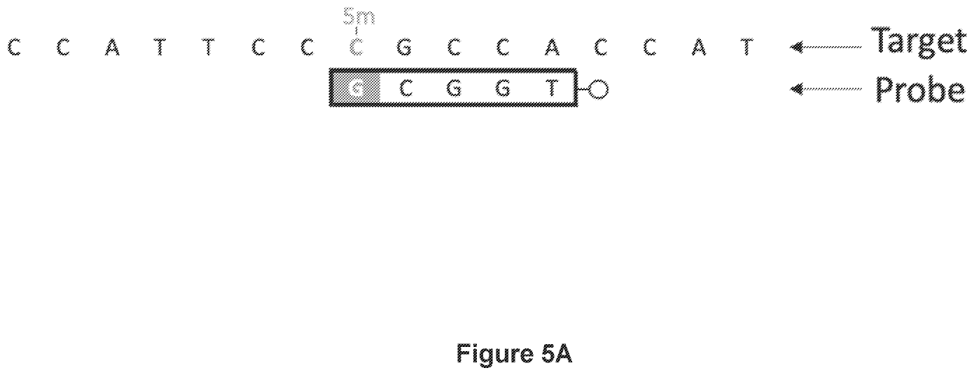

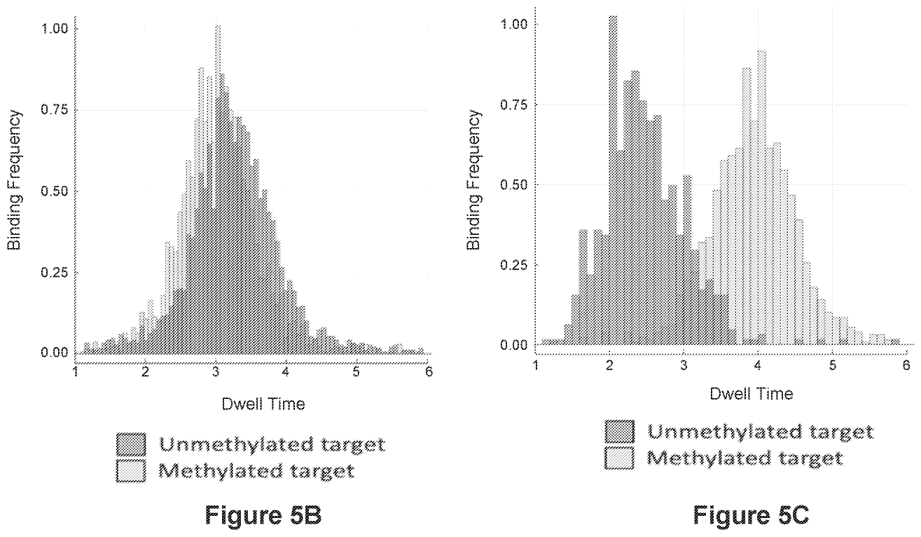

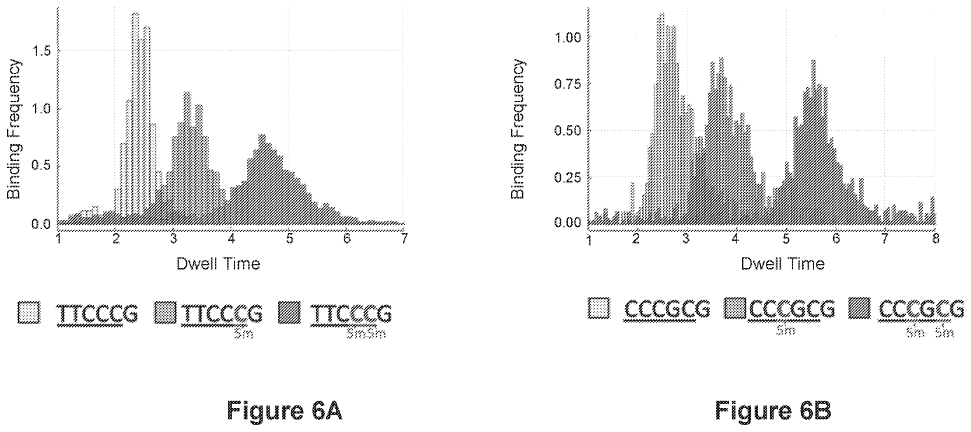

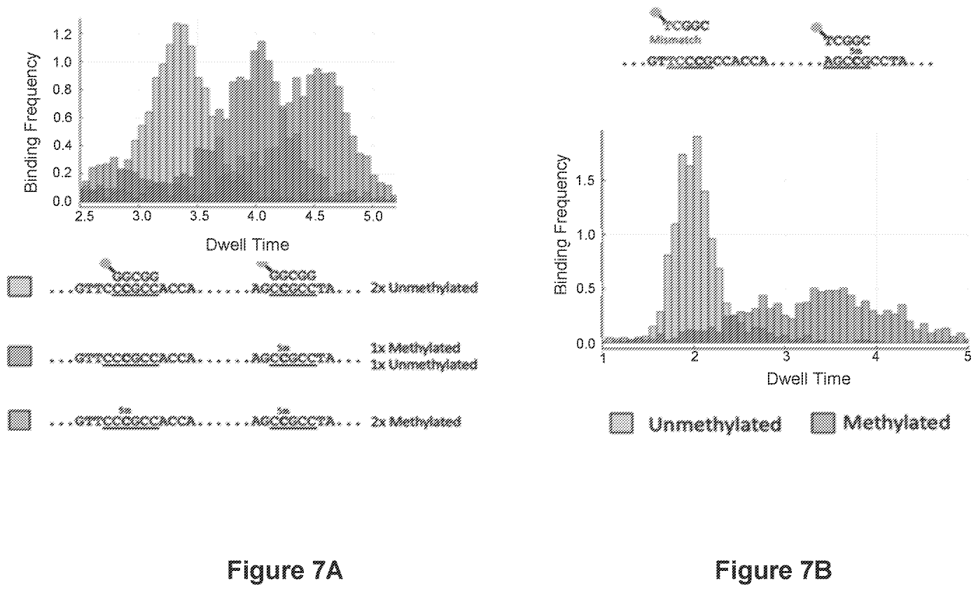

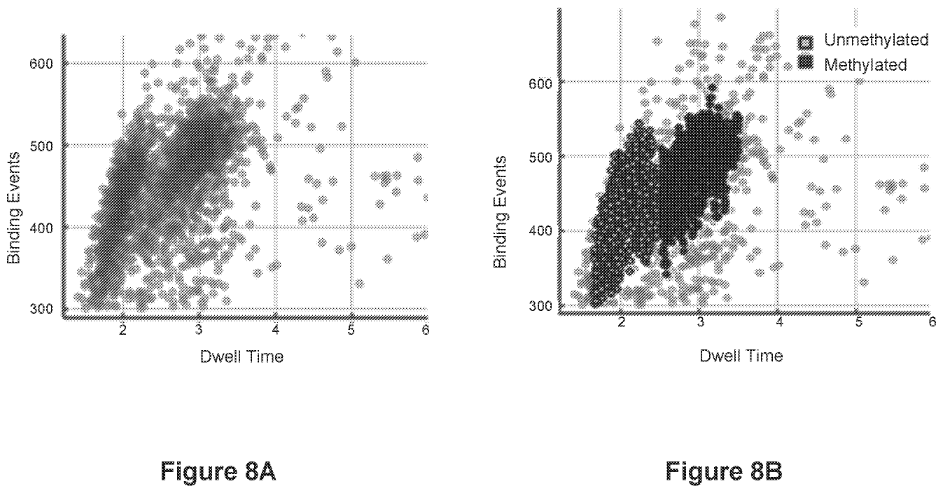

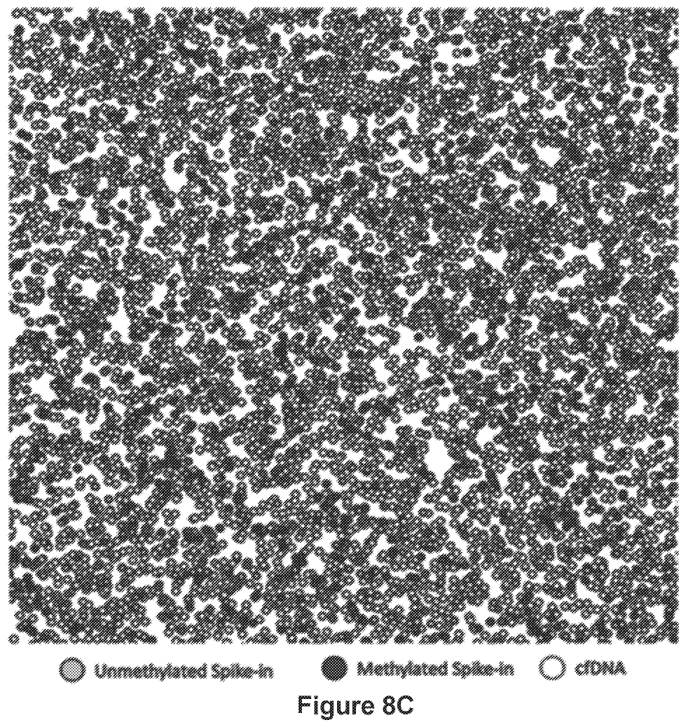

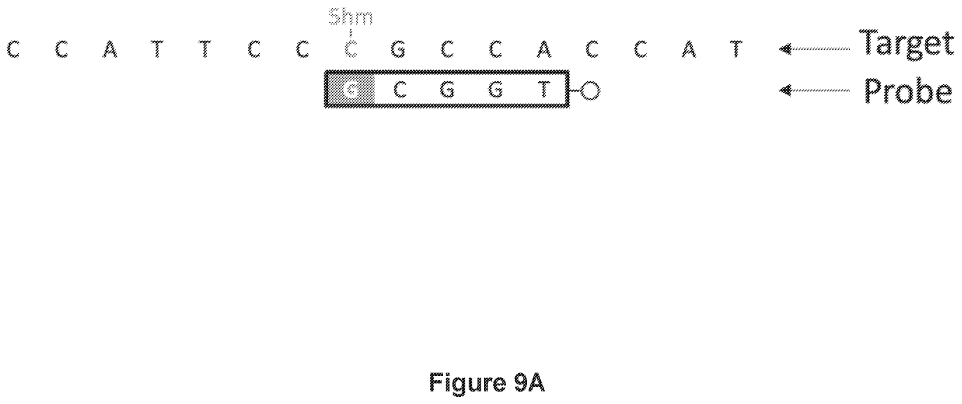

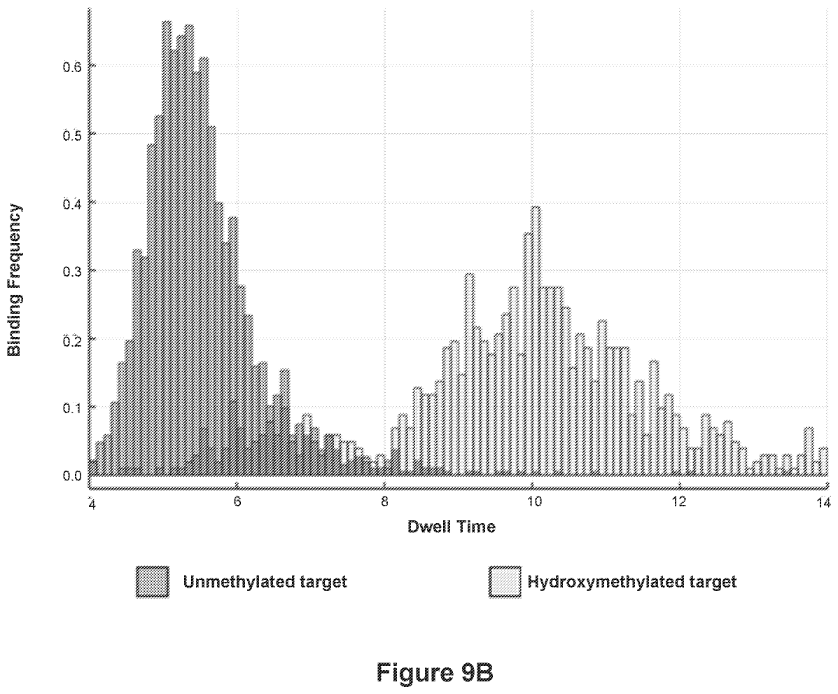

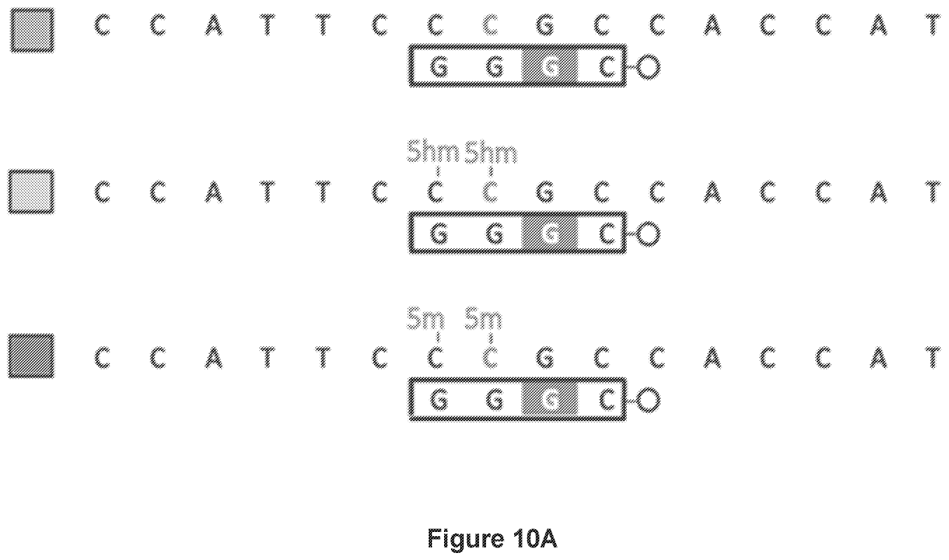

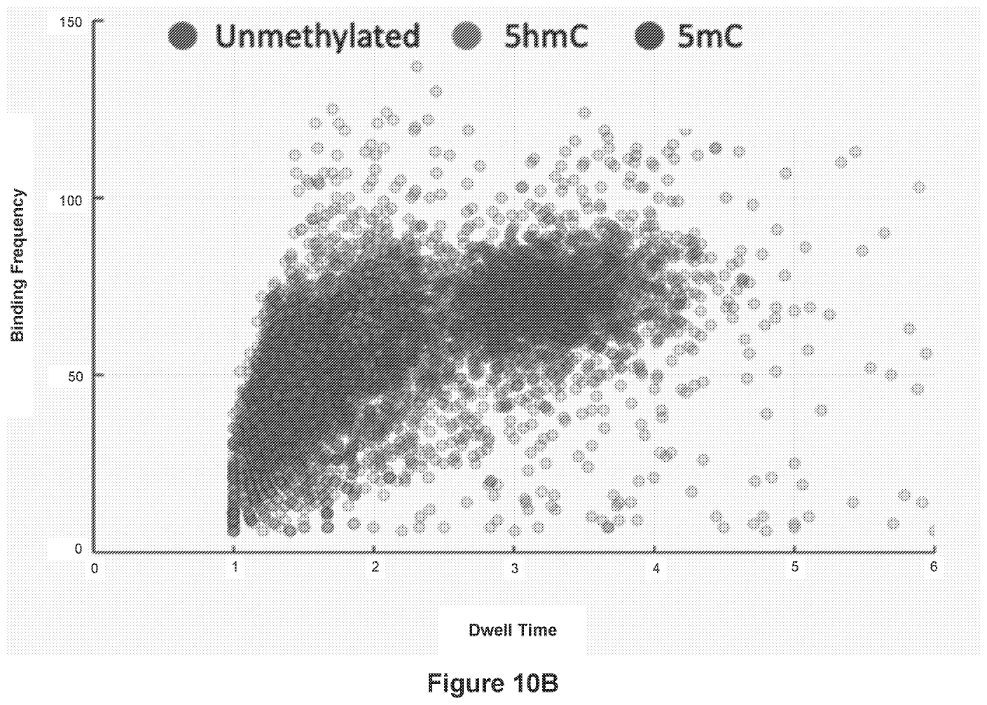

The data was processed using a drift correction algorithm, a single molecule localization algorithm and algorithms that determined the number of repeat binding events, the dark time and the bright time of binding for each individual molecule (e.g., model 1924 of control module 1922 of FIG. 11). Statistics on dwell times for oligos targeting target 1 (non-methylated) and target 2 (methylated) were compared and the data is summarized in FIG. 3A through 5B.

### Example 2: Detection of Methylated Status from a FFPE Tumor Sample

Eight FFPE sections 5 μm thick with a service area of 250 mm2 were obtained from a tumor sample from a lung cancer patient. DNA was isolated using the QIAamp DNA FFPE tissue kit (following manufactures instructions). The DNA was then fragmented to ˜150 bp using the ME220 Focused-ultrasonicator (Covaris). Biotin labelling of the DNA was then performed using the following terminal transferase (TdT) reaction: 200 ng cfDNA, 1× TdT reaction buffer, 4 uM ddATP-Biotin, 250 nM CoC12, 40U TdT, and incubated for 90 minutes. Samples were then purified using GeneJet PCR purification kit following manufacturer's instructions. A proprietary pool of control oligonucleotides were spiked into the sample; the pool of oligonucleotides comprise methylated and non-methylated cytosines.

A custom flow cell was prepared using a streptavidin coated cover glass (Schott AG, Mainz Germany)) attached to plastic flow cell chamber (Sticky-Slide VIV 0.4, Ibidi, Martinstried, Germany). The flow cell was washed with BX (5 mM Tris, 10 mM MgCL2, 0.05% Tween 20 and 1 mM EDTA). Biotin labelled tumor DNA prepared by the TdT protocol described above, was added to a single channel of the flow cell. To render the DNA single stranded and clean the flow cell, the channel is washed 5 times with freshly prepared 0.5M NaOH, including one two-minute incubation in NaOH at room temperature, before washing with Bx buffer 4 times. 5 nM of identification oligonucleotides (for a genome-wide analysis up to 250 probes chosen randomly from the complete repertoire of 5mers are tested) in BX+F (5 mM Tris, 10 mM MgCL2, 0.05% Tween 20 and 1 mM EDTA, 30% Formamide) was added to each flow cell. The set of oligos from the complete 1024 repertoire of 5mers were added sequentially. Washing between oligonucleotide imaging was performed with 150 ul BX+F three times. Imaging was performed on the ONI Nanoimager S in TIRF mode. A total of 500 frames with 200 ms per frame was captured per oligo. After molecule identification, methylation probes (see table 1) in BX+F (5 mM Tris, 10 mM MgCL2, 0.05% Tween 20 and 1 mM EDTA, 30% Formamide) were added to each flow cell sequentially. Imaging was performed on the ONI Nanoimager S in TIRF mode with the focus lock activated and 10% laser (532 nm) power. A total of 2000 frames with 200 ms per frame was captured per oligo.

The data was processed using a drift correction algorithm, a single molecule localization algorithm and algorithms that determined the number of repeat binding events, the dark time and the bright time of fluorescence signal due to binding for each individual molecule (e.g., one or more models 1924 of control module 1922 of FIG. 11). The resulting processed data was further processed using a statistical algorithm which estimates the identity of the molecule by reference to a cancer database and then provides a likelihood for methylation of all sites containing cytosine, C residues, based on measured dwell times of sample DNA and control DNA. As an alternative a machine learning algorithm is used to classify the molecules as bearing methylated Cs or not.

### Example 3: Whole Genome Methylation Assay for Plasma DNA

To detect methylation status of cell free DNA in blood plasma, the following steps are carried out.
(i) Centrifuge blood sample to obtain plasma.
(ii) Purify cfDNA from plasma using commercial kit (e.g. ThermoFisher MagMax Cell-Free DNA Isolation kit). (CfDNA must be in an EDTA-free buffer for subsequent steps—elute DNA using an EDTA free buffer from the kit or else exchange the buffer (e.g. using a commercial PCR purification kit such as GeneJet PCR purification kit).
(ii) Biotin labeling is carried out by incubating DNA with terminal transferase (TdT) and ddATP biotin in TdT buffer.
(iv) Purify samples using a commercial DNA purification kit such as Genejet PCR purification kit. (Biotinylated control DNA may be spiked in at this point—or unbiotinylated DNA earlier in the process).
(v) A custom flow cell is prepared using a streptavidin coated cover glass (Schott AG, Mainz Germany)) attached to plastic flow cell chamber (Sticky-Slide VIV 0.4, Ibidi, Martinstried, Germany).
(vi) The flow cell is washed with PBS and Bx Buffer.
(vii) Biotin labelled DNA prepared above is added to a single channel of the flow cell. After 4 min the flow cell was washed with 150 ul of Bx, 4 times.
(viii) To render the DNA single stranded and clean the flow cell, the channel is washed 5 times with freshly prepared 0.5M NaOH, including one two-minute incubation in NaOH at room temperature, before washing with Bx buffer 4 times.
(ix) The flow cell is mounted on the ONI nanoimager.
(x) The channel is primed with imaging buffer appropriate to the first round of oligos, then the fluorescently labelled oligos are added to the channel.
(xi) Imaging is performed in TIRF mode with 200 ms frames. Multiple fields of view may be collected for each round of imaging.
(xii) Further rounds of oligos are passed through the flow cell; up to 1024 5mers may be passed through, for example. Between subsequent rounds of imaging oligos, the channel is washed 2× with wash buffer;
(xiii) After all rounds of fluorescently labelled oligos, the pattern of probes binding and not binding for each DNA fragment on the flow cell is compared to a reference genome to identify the position of the DNA in the genome and the methylation pattern along the identified fragment.
(xiv) The binding kinetics of probes (e.g. bright time, dark time, number of repeat binding events) is used to determine the methylation status of cytosines in each fragment.

The following is a listing a sequence of methyl detection probes (all combinations of probes capable of binding a CpG motif): CCCCG; CCCGG; CCGGG; CGCCC; CCGCC; CCCGC; GCCCG; CGGGG; CCCGT; CGGCC; CCGGC; GGCCG; GCCGG; CCCGA; CGCCG; CCGCG; CCGGT; CCGGA; CGGGC; GGGCG; GGCGG; GCGGG; GCGCC; GCCGC; CGGCG; CGCGG; CGGGT; CGCCA; CCGCA; GCCGT; TCCCG; CGGGA; GGCGC; GCGGC; GCCGA; ACCCG; TGCCG; AGCCG; CGCCT; CCGCT; CGCGC; GCGCG; CGGCA; GGCGT; GCGGT; GGCGA; GCGGA; TCCGG; ACCGG; TGGCG; TGCGG; CGCGT; CCGTG; CGGCT; AGGCG; AGCGG; CGCGA; GCGCA; CCGAG; TCGCC; TCCGC; TCGGG; ACGCC; ACCGC; GCGCT; TGCGC; AGCGC; CGGTG; CGTGG; ACGGG; CGGAG; CGAGG; TCCGT; ACCGT; AGCGT; TGCGT; CCGTC; CGTCC; GTCCG; TCGGC; TCCGA; CGTGC; GCGTG; GTGCG; ACGGC; ACCGA; AGCGA; CCACG; CACCG; TGCGA; CGCAG; CAGCG; CCGTA; CCGAC; CGACC; CGAGC; GCGAG; GACCG; GAGCG; TCGCG; CCTCG; CTCCG; ACGCG; TCGGT; CGTGT; CGCTG; CTGCG; ACGGT; CCGAT; CGGTC; GGTCG; GTCGG; CGAGT; CGTGA; TCGGA; ACGGA; CACGG; CGGTA; CCGTT; CGTCG; CGAGA; CGGAC; GGACG; GACGG; ACGCA; TCGCA; CTCGG; GCGTC; GTCGC; CGGAT; CGCAC; CACGC; CGACG; GCACG; GCGTA; ACGCT; TCGCT; GTCGT; GCGAC; GACGC; CGCAT; CGCTC; CTCGC; GCTCG; CGGTT; CACGT; CGTCA; GCGAT; CGCTA; CCGAA; GTCGA; GACGT; CGTCT; CTCGT; TAGCG; CGACA; CACGA; ATCCG; GCGTT; TACCG; ATGCG; AGTCG; GACGA; ACGTG; TCGTG; TGTCG; CGACT; CTCGA; ACGAG; AGACG; CGCTT; CGTAG; TCGAG; CGGAA; TGACG; ATCGG; CGCAA; TACGG; TTCCG; TTGCG; GCGAA; CGATG; ATCGC; TACGC; AAGCG; ACGTC; AACCG; CGTTG; TCGTC; ATCGT; ACGTA; TACGT; ACACG; TCGTA; TCACG; CGTAC; GTACG; TTCGG; ACGAC; CGTAT; ACGAT; TCGAC; ACTCG; ATCGA; TCGAT; CATCG; TCTCG; TACGA; TTCGC; CGATC; GATCG; ACGTT; AACGG; CGAAG; CTACG; CGATA; TCGTT; TTCGT; AACGC; CGTTC; GTTCG; CGTTA; AACGT; TTCGA; CGATT; CTTCG; CGTAA; CAACG; ACGAA; AACGA; CGTTT; CGAAT; TCGAA; CGAAC; GAACG; ATACG; TATCG; ATTCG; TTACG; CGAAA; AATCG; TAACG; TTTCG; and AAACG.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

The claims are amended as follows:

1. A method for determining the identity and modification status of a nucleic acid molecule, the method comprising:
a. fixing the nucleic acid on a surface, thus obtaining a nucleic acid attached at the surface;
b. exposing one or more oligos of known sequence to the nucleic acid, one or more or a combination of said oligos capable of determining the identity of said nucleic acid and detecting the binding of one or more of said oligos to the nucleic acid and determining the identity of the said nucleic acid;
c. exposing one or more oligos of known sequence to the nucleic acid molecule, one or more of said oligos capable of binding differently to a sequence when the sequence is modified compared to when the sequence is not modified and detecting the binding of the oligos to the nucleic acid and measuring a binding characteristic of the oligos; and
d. assigning modification status to the molecule of determined identity by assessing the signature of the measured characteristic.

2. A method for determining the modification status of a nucleic acid comprising:
a. fixing the nucleic acid on a surface, thus obtaining a nucleic acid at a fixed location on the surface;
b. exposing one or more oligos of known sequence to the nucleic acid molecule, one or more of said oligos capable of having a different binding profile when the sequence is modified compared to when the sequence is not modified;
c. detecting the binding of the oligos to the nucleic acid and determining if the binding profile better matches the binding profile of when the sequence is modified or the binding profile of when the sequence is not modified; and
d. assigning modification status to the nucleic acid molecule or one or more locations on the nucleic acid molecule.

3. A method of determining a sequence and epi-sequence of at least a portion of a nucleic acid molecule, comprising:
(a) fixing the nucleic acid molecule on a test substrate when the nucleic acid molecule is a single stranded molecule or denaturing the nucleic acid molecule to a single stranded molecule and fixing the single stranded nucleic acid molecule on the test substrate when the nucleic acid molecule is a double stranded molecule or fixing the nucleic acid molecule on the test substrate and denaturing the nucleic acid molecule on the test substrate to a single stranded molecule when the nucleic acid molecule is a double stranded molecule, thereby forming a fixed single stranded nucleic acid on the test substrate;
(b) exposing the fixed single stranded nucleic acid to a respective oligonucleotide probe species in a set of oligonucleotide probe species, wherein each respective oligonucleotide probe species of the set of oligonucleotide probe species is capable of hybridizing to its complementary portion located at one or more locations on the fixed single stranded nucleic acid and has:
(i) a unique respective predetermined sequence,
(ii) a predetermined length, and
(iii) a respective label selected from the group consisting of a dye, a fluorescent nanoparticle, a plasmon resonant particle, a light-scattering particle, a nanoparticle, and a fluorescence resonance energy transfer (FRET) partner which is capable of producing a fluorescent signal,
wherein the exposing step occurs under conditions such that:
(i) oligonucleotide probes of the respective oligonucleotide probe species of the set of oligonucleotide probe species repetitively transiently and reversibly bind to the one or more locations on the fixed single stranded nucleic acid on the test substrate, thereby forming a respective transient heteroduplex on each of the one or more locations on the fixed single stranded nucleic acid on the test substrate, and
(ii) respective instances of optical activity from the respective label are generated and detected by repetitively transiently and reversibly binding the oligonucleotide probes of the respective oligonucleotide probe species of the set of oligonucleotide probe species to the one or more locations on the fixed single stranded nucleic acid on the test substrate;
(c) determining if one or more portions of the fixed single stranded nucleic acid are complementary to the respective oligonucleotide probe species of the set of oligonucleotide probe species by measuring the respective instances of optical activity on each of the one or more locations on the fixed single stranded nucleic acid on the test substrate occurring during the exposing step using a two-dimensional imager capable of detecting the respective instances of optical activity generated from the respective label, thereby obtaining a first set of one or more positions on the fixed single stranded nucleic acid that are complementary to the respective oligonucleotide probe species of the set of oligonucleotide probe species;
(d) washing the test substrate to remove the respective oligonucleotide probe species of the set of oligonucleotide probe species from the test substrate;
(e) repeating steps (b)-(d) by exposing the fixed single stranded nucleic acid on the test substrate to another respective oligonucleotide probe species in the set of oligonucleotide probe species, thereby obtaining a second set of one or more positions on the fixed single stranded nucleic acid that are complementary to another respective oligonucleotide probe species in the set of oligonucleotide probe species;
(f) determining the sequence of at least the portion of the nucleic acid based at least in part on the first set of one or more positions on the fixed single stranded nucleic acid that are complementary to the respective oligonucleotide probe species of the set of oligonucleotide probe species and the second set of one or more positions on the fixed single stranded nucleic acid that are complementary to the another respective oligonucleotide probe species of the set of oligonucleotide probe species; and
(g) determining whether the portion of the nucleic acid molecule has an epigenetic modification based on an observed differential binding behavior of the oligonucleotide probes of the respective oligonucleotide probe species in the set of oligonucleotide probe species to their complementary portion located at one or more locations on the fixed single stranded nucleic acid when the one or more locations has an epigenetic modification compared to when the one or more locations does not have an epigenetic modification.

4. The method of claim 1, wherein the binding of one or more oligos is transient and each site on each target molecule is capable of being bound transiently multiple times.

5. The method of claim 1 or 4, wherein the binding difference of the oligonucleotide to the nucleic acid is measured as a function of an on-time and off-time, and/or the fluorescence intensity of the signal.

6. The method of claim 1 or 3, wherein the same oligos are able to determine identity and determine modification status.

7. The method of claim 1 or 3, wherein said identity of a nucleic acid comprises its genomic origin.

8. The method of claim 1 or 3, wherein the determining of identity is done by comparing to a database comprising matching the obtained pattern of binding to an in silico pattern of binding for segments of the genome.

9. The method of any one of claims 1-8, wherein the modification is a chemical modification comprising 5-methylcytosine (5mC), 5-hydroxymethylcytosine (5hmC), 5-formylcytosine (5fC), 5-carboxylcytosine (5caC), and N6-methadenine (6 mA) or any other modification common in nucleic acids found in biological organisms.

10. The method of any one of claims 1-9, wherein the modification is due to DNA damage.

11. The method of any one of claims 1-10, wherein spiked-in controls are used as a reference.

12. The method of any one of claims 1-11, wherein the nucleic acid is subjected to a treatment to alter the modification prior to binding of the oligonucleotide.

13. The method of any one of claims 1-12, wherein the target is elongated and the position of modification along its length is localized.

14. The method of any one of claims 1-13, wherein the molecules are arrayed at high density and super-resolution is used to resolve individual molecules.

15. The method of any one of claims 1-14, wherein the number of modifications on the molecule are enumerated or estimated.

16. The method of claim 3, wherein the binding profiles of each oligonucleotide capable of binding to a nucleic acid sequence that may bear a modification are characterized by testing against synthetic modified and non-modified versions of nucleic acid sequences that may bear a modification and thus serve as a reference to compare the binding profiles obtained for the sample molecules.

17. The method of any one of claims 1-16, wherein the oligonucleotide comprises a labelled oligonucleotide, and wherein the label comprises one or more fluorophores, nanoparticles, proteins, or nanostructures.

18. The method of any one of claims 1-17, wherein the one or more oligos are <=8, <=, <=6, <=5, <=4, or <=3 nucleotides in length, and wherein the oligos optionally comprise one or more modifications comprising LNA residues, a degenerate or universal nucleotide position, Uaq cap, or pyrene cap.

19. The method of any one of claims 1-18, wherein machine learning is employed to analyze binding data from multiple oligonucleotides to determine the modification status and/or identity of the nucleic acid molecule.

20. The method of any one of claims 1-19, wherein the nucleic acid is a cell-free nucleic acid molecule.

2-35. (canceled)

37-41. (canceled)

43-44. (canceled)


SEQUENCE LISTING (6 sequences)

SEQ ID NO: 1 - DNA, 21 bp, synthetic construct
  misc_feature 1..21: note = Synthetic
  source 1..21: mol_type = other DNA
cggatcgatcgatgtcgttga

SEQ ID NO: 2 - DNA, 14 bp, synthetic construct
  misc_feature 1..14: note = Synthetic
  source 1..14: mol_type = other DNA
gtagctgcgtgcta

SEQ ID NO: 3 - DNA, 16 bp, synthetic construct
  misc_feature 1..16: note = Synthetic
  source 1..16: mol_type = other DNA
ccattcccgccaccat

SEQ ID NO: 4 - DNA, 13 bp, synthetic construct
  misc_feature 1..13: note = Synthetic
  source 1..13: mol_type = other DNA
gttcccgccacca

SEQ ID NO: 5 - DNA, 91 bp, synthetic construct
  misc_feature 1..91: note = Synthetic
  source 1..91: mol_type = other DNA
tttttttttttttttttttttttttttttccattcccgccaccatcgcctcaatccctgtgcgctaatttttttttttttttttttttttt

SEQ ID NO: 6 - DNA, 91 bp, synthetic construct; identical to SEQ ID NO: 5
  misc_feature 1..91: note = Synthetic
  source 1..91: mol_type = other DNA